(19) United States
(12) Patent Application Publication
(10) Pub. No.: US 2003/0083520 A1
(43) Pub. Date: May 1, 2003

(54) Process for preparing bicyclic amino acid

(75) Inventors: David Clive Blakemore, Sandwich (GB); Justin Stephen Bryans, Sandwich (GB)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/137,913

(22) Filed: May 2, 2002

(30) Foreign Application Priority Data
May 4, 2001 (GB) 0110935.4

(51) Int. Cl. C07C 229/32

(52) U.S. Cl. 562/500; 560/354

(57) ABSTRACT

The invention relates to a process for preparing (1α,3α,5α)(3-aminomethyl-bicyclo[3.2.0]hept-3-yl)-acetic acid, or an acid addition salt thereof. An embodiment of the process is shown in the following reaction scheme:

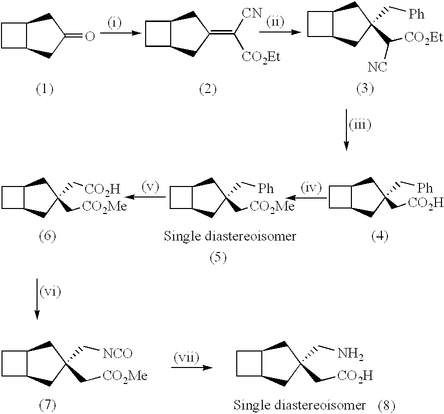

### FIELD OF THE INVENTION

This invention relates to a process for preparing a bicyclic amino acid, and more particularly to a process for preparing (1α,3α,5α)-(3-aminomethyl-bicyclo[3.2.0]hept-3-yl)-acetic acid, or an acid addition salt thereof.

### BACKGROUND TO THE INVENTION

Gabapentin (Neurontin®) is an anticonvulsant agent that is useful in the treatment of epilepsy and that has recently been shown to be a potential treatment for neurogenic pain. It is 1-(aminomethyl)-cyclohexylacetic acid of structural formula:

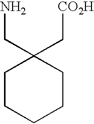

U.S. Patent Application No. 60/160725 describes a series of novel bicyclic amino acids which are analogues of gabapentin, their pharmaceutically acceptable salts, and their prodrugs of formulae:

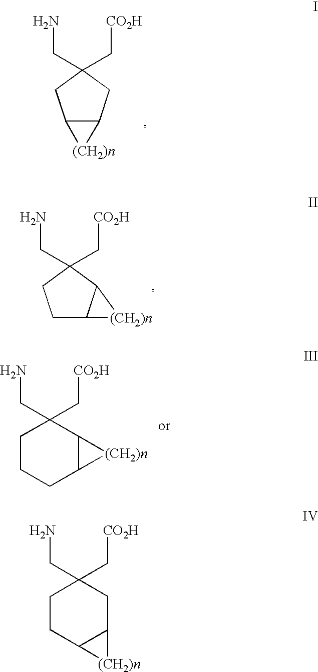

wherein n is an integer of from 1 to 4. Where there are stereocenters, each center may be independently R or S, preferred compounds being those of Formulae I-IV above in which n is an integer of from 2 to 4. The compounds are disclosed as being useful in treating a variety of disorders including epilepsy, faintness attacks, hypokinesia, cranial disorders, neurodegenerative disorders, depression, anxiety, panic, pain, neuropathological disorders, and sleep disorders. Certain of the compounds disclosed in that patent application have high activity as measured in a radioligand binding assay using [³H]gabapentin and the α₂δ subunit derived from porcine brain tissue (Gee N. S., Brown J. P., Dissanayake V. U. K., Offord J., Thurlow R., Woodruff G. N., J. Biol. Chem., 1996;271:5879-5776). Results for some of the compounds are set out in the following table:

TABLE 1

| | | α₂δ |
|---|---|---|
| | | binding |
| | | affinity |
| Compound | Structure | (μM) |
| (1α,3α,5α)(3-Aminomethyl- bicyclo[3.2.0]hept-3-y1)-acetic acid | 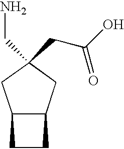 | 0.038 |
| (+/−)-(1α,5β)(3-Aminomethyl- bicyclo[3.2.0]hept-3-yl)-acetic acid | 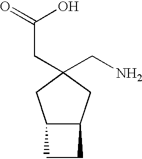 | 2.86 |
| (1α,3β,5α)(3-Aminomethyl- bicyclo[3.2.0]hept-3-yl)-acetic acid | 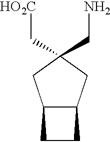 | 0.332 |

The present invention is concerned with the production of the active compound (1α,3α,5α)(3-aminomethyl-bicyclo[3.2.0]hept-3-yl)-acetic acid, or an acid addition salt thereof. The synthetic route described in U.S. No. 60/160725 proceeds via a nitro derivative produced using nitromethane, and results in a 95:5 mixture of diastereoisomers (1α,3α,5α) and (1α,3β,5α) respectively. The present invention addresses the problem of obtaining an improved yield of product and producing a single diastereomeric product. This problem is solved by the process defined below.

### SUMMARY OF THE INVENTION

The present invention provides a process for preparing (1α,3α,5α)(3-aminomethyl-bicyclo[3.2.0]hept-3-yl)-acetic acid, or an acid addition salt thereof, which comprises the following steps:

(i) condensing a cyclic ketone (1) with an alkyl cyanoacetate to form a cyanoester (2):

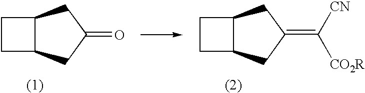

in which R is an alkyl group having 1 to 6 carbon atoms;

(ii) reacting the cyanoester (2) with an arylalkyl or alkenyl Grignard reagent to form a cyanoester (3):

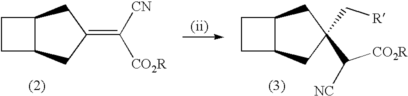

in which R′ is a phenyl or phenyl-C₁-C₄ alkyl group or a C₂-C₆ alkenyl group;

(iii) removing the cyano group of the cyanoester (3) by reaction with a base to form a carboxylic acid (4):

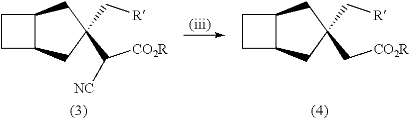

(iv) converting the carboxylic acid (4) to its alkyl ester (5):

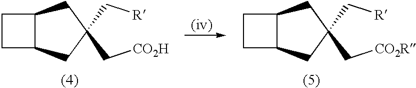

in which R″ is an alkyl group having 1 to 6 carbon atoms;

(v) oxidising the alkyl ester (5) to form the acid (6): (5) (6)

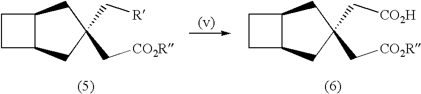

(vi) converting the carboxylic acid group of the acid (6) to an isocyanate group, thereby forming the compound (7):

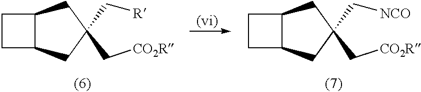

and

(vii) hydrolysing the isocyanate and ester groups of compound (7) to form the desired compound (1α,3α,5α)(3-aminomethyl-bicyclo[3.2.0]hept-3-yl)-acetic acid (8), or an acid addition salt thereof:

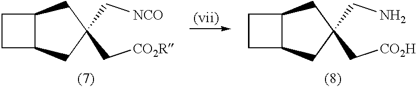

### DETAILED DESCRIPTION

The starting material in the process of the invention is the cyclic ketone of formula (1). Our copending application GB 0110884.4, filed May 4, 2001, the disclosure of which is hereby incorporated by reference, describes a process for preparing this cyclic ketone according to the following reaction Scheme 1:

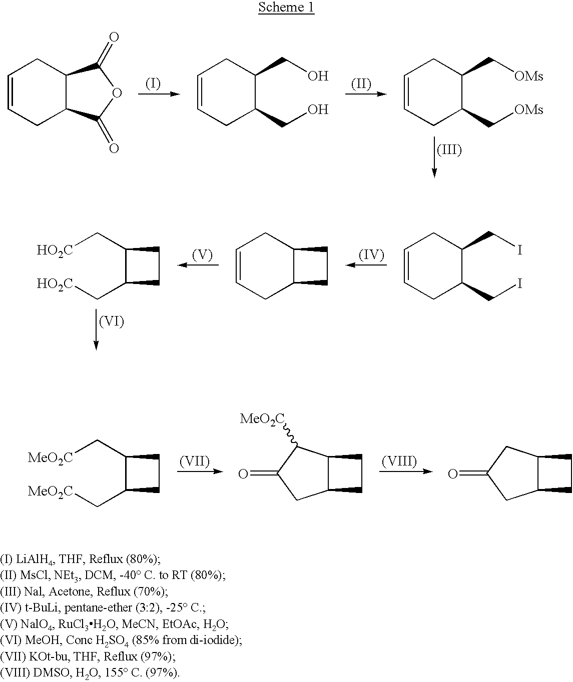

Another method of preparing the cyclic ketone (1) is disclosed in U.S. No. 60/160725 and is reproduced below in Reference Example 1.

In step (i) of the process according to the invention (cf. Scheme 2 below), the ketone (1) is condensed with an alkyl cyanoacetate, for example ethyl cyanoacetate, preferably in an organic solvent such as toluene, benzene, xylenes or n-heptane, to which acetic acid and β-alanine or ammonium acetate, or piperidine are added.

Step (ii) involves the use of an arylalkyl or alkenyl Grignard reagent, and results in the production of a 1:1 mixture of diastereomeric cyanoesters (3). The arylalkyl Grignard reagent is preferably a benzyl Grignard reagent, such as benzylmagnesium chloride, benzylmagnesium bromide or benzylmagnesium iodide. Reaction with the arylalkyl Grignard reagent can be carried out at a temperature from −100° C. to 110° C., generally at room temperature.

The alkenyl Grignard reagent which may be used in step (ii) is preferably a vinyl, allyl or 2-butenyl Grignard reagent, such as vinylmagnesium chloride, vinylmagnesium bromide, allylmagnesium chloride, allylmagnesium bromide or 2-butenylmagnesium chloride. An organometallic reagent such as vinyl lithium can similarly be used. The reaction of step (ii) with an alkenyl Grignard reagent is preferably carried out in the presence of a dialkylzinc, such as dimethyl zinc, or a copper (I) salt, such as copper (I) iodide or copper (I) cyanide. This reaction is preferably carried out with cooling, for example at a temperature of from −100° C. to 0° C.

In step (iii) the cyanoester (3) is reacted with a base to remove the cyano group and hydrolyse the ester group, resulting in the single diastereomeric acid (4). The base used may be an alkali metal hydroxide, such as potassium hydroxide, sodium hydroxide, lithium hydroxide or cesium hydroxide. The reaction may be carried out in a solvent such as ethylene glycol, 2-methoxyethyl ether, 1,4-dioxane or diethylene glycol.

The carboxylic acid group of acid (4) is protected by conversion to its alkyl ester (5). The alkyl ester is preferably a methyl ester, and to obtain this the acid (53) may be added

to a mixture of iodomethane in a solvent selected from dichloromethane, chloroform, tetrahydrofuran, toluene or 1,4-dioxane to which a base such as 1,8-diazabicyclo[5.4.0]undec-7-ene (DBU), triethylamine or 1,5-diazabicyclo[4.3.0]non-5-ene (DBN) is added and stirred at a temperature from −40° C. to 110° C.; or

to a mixture of methanol and a concentrated acid such as sulphuric acid or hydrochloric acid at a temperature ranging from 0° C. to 100°0 C.; or

to trimethylsilyldiazomethane and methanol in benzene or toluene at a temperature from −40° C. to 100° C.; or

to diazomethane in a solvent such as benzene, toluene, dichloromethane at a temperature from −40° C. to 40° C.

In step (v) the aryl, e.g. phenyl, group or the alkenyl, e.g. allyl, group of the resulting ester (5) is oxidized to a carboxylic acid group, for example by treatment with sodium periodate and ruthenium (III) chloride in a mixture of carbon tetrachloride or ethyl acetate and acetonitrile to which water is added. The mixture is stirred at a temperature from −40° C. to 80° C. to give the carboxylic acid (6).

In step (vi) the carboxylic acid group of acid (6) is converted to isocyanate by addition

to a mixture of a base selected from triethylamine or diisopropylethylamine and a solvent selected from toluene, benzene, xylenes, tetrahydrofuran, diethyl ether or n-heptane to which diphenylphosphoryl azide (DPPA) is added and stirring at a temperature from 0° C. to 150° C. to produce the isocyanate of formula (7); or to ethyl chloroformate or isobutyl chloroformate and a base such as triethylamine or diisopropylethylamine in tetrahydrofuran or acetone or diethyl ether at a temperature of −40° C. to 78° C. followed by addition of sodium azide in water and tetrahydrofuran or acetone followed by addition of toluene or benzene and refluxing.

In step (vii), the isocyanate and ester groups of compound (7) are simultaneously hydrolysed to amino and carboxylic acid groups, e.g. by aqueous hydrochloric acid at a concentration of from 0.01 M to 12 M optionally in the presence of a solvent such as 1,4-dioxane, acetic acid or water to produce the amino acid (8).

An embodiment of the invention using a benzyl Grignard reagent in step (ii) is detailed below (“benzyl route”). The main advantage of this route is that the addition of the benzyl Grignard reagent BnMgCl can be carried out at room temperature without the necessity for an additive (such as dimethylzinc or copper (I) cyanide). The benzyl Grignard addition also appears to be stereoselective (there being no evidence from NMR or GC analysis for the presence of more than one diastereoisomer of the benzyl acid after hydrolysis of the cyanoester).

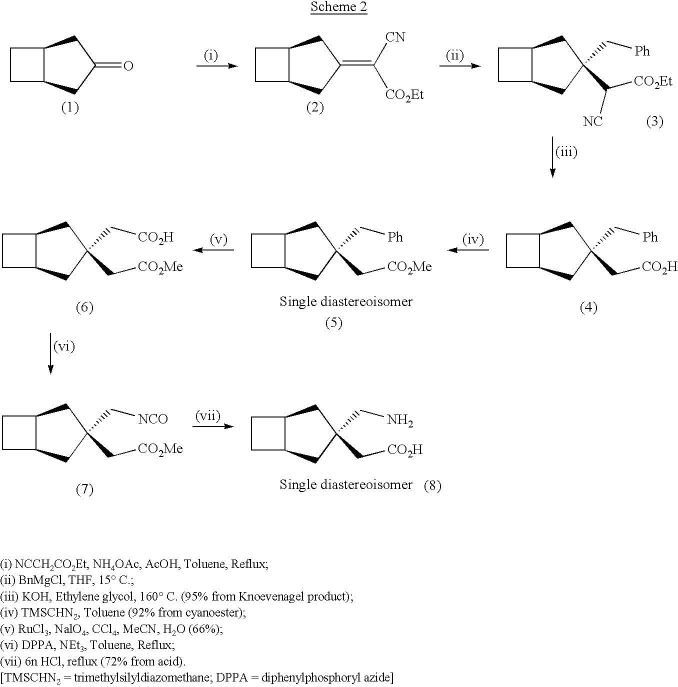

An embodiment of part of the process of the invention in which an allyl Grignard reagent is used in step (ii) is detailed in Schemes 3 and 4 below (“allyl route”). The main advantage of this route is that the allyl oxidation (to the carboxylic acid) requires only four equivalents of sodium periodate in addition to the ruthenium trichloride. The main disadvantage of this route is that the conjugate addition of the allyl Grignard requires an additive such as dimethylzinc or copper (I) cyanide. The yields obtained with dimethylzinc over the two steps of conjugate addition and hydrolysis were higher than with the cuprate addition (89% as opposed to 70%).

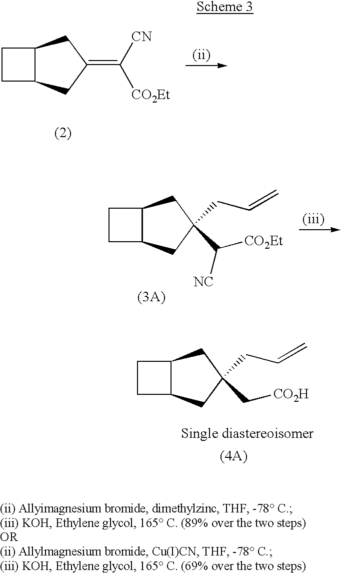

The copper (I) cyanide reaction was investigated at two different temperatures and while the reaction appears to go cleanly at 0° C. (apparently giving a single diastereoisomer after hydrolysis) the yield was poorer than at −78° C. (presumably due to polymerisation). However, other temperatures may be used.

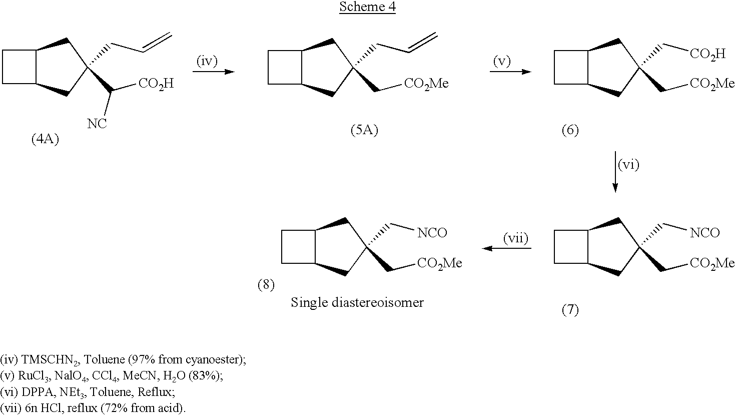

The invention is illustrated by the following Examples.

### EXAMPLES

### Reference Example 1

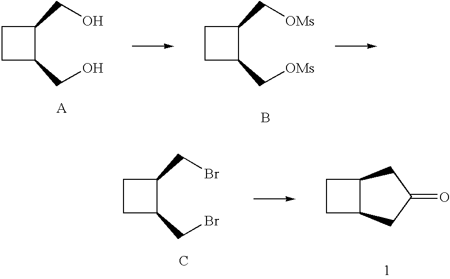

Synthesis of Compound A

Lithium aluminum hydride (69.4 mL of a 1 M solution in ether, 69.4 mmol) was added dropwise to a stirring solution of cis-cyclobutane-1,2-dicarboxylic acid (5 g, 34.7 mmol) in THF (60 mL) at 0° C. under argon. The mixture was allowed to warm to room temperature and stirred for 16 hours. The mixture was cooled to 0° C. and quenched by careful addition of water (2.7 mL), sodium hydroxide solution (2.7 mL of a 15% w/v solution), and water (8.1 mL). The mixture was stirred for 15 minutes, and the precipitate was removed by filtration. The solvent was evaporated under reduced pressure to give the alcohol A as a colorless oil (4.0 g, 98%); δ_{H} (400 MHz; CDCl₃): 3.85 (2H, m), 3.6 (2H, m), 3.2 (2H, s), 2.7 (2H, m), 2 (2H, m); 1.55 (2H, m); δ_{c} (400 MHz; CDCl₃): 63.15, 37.83, 20.40.

Synthesis of Compound B

Mesyl chloride (6.2 mL, 79.1 mmol) was added dropwise to a stirring solution of A (4.0 g, 34.4 mmol) in dichloromethane (150 mL) at −40° C. under argon. Triethylamine (12.0 mL, 86.0 mmol) was then added dropwise, and the mixture was allowed to warm slowly to room temperature. After stirring for 16 hours, the mixture was quenched by addition of dilute hydrochloric acid (50 mL). The organic layer was separated, and the aqueous layer was further extracted with dichloromethane (2×50 mL). The combined organic fractions were washed with brine, dried (MgSO₄), and the solvent was evaporated under reduced pressure. The residue was chromatographed (SiO₂, heptane/ethyl acetate, 6:4) to give the mesylate B (6.1 g, 73%) as a white solid; R_{f} (heptane/ethyl acetate, 1:1) 0.18. δ_{H} (400 MHz; CDCl₃): 4.3 (4H, m), 3.05 (6H, s), 2.9 (2H, m), 2.2 (2H, m), 1.8 (2H, m); δ_{c}(400 MHz; CDCl₃): 69.51, 37.45, 35.28, 21.09.

Synthesis of Compound C

Anhydrous lithium bromide (10.6 g, 121.8 mmol) was added to a stirring mixture of B (5.95 g, 24.4 mmol) in acetone (50 mL) under argon and the mixture was refluxed for 2 hours. After cooling, the acetone was evaporated under reduced pressure and the residue was taken up in ether (50 mL), washed with water (50 mL), brine, dried (MgSO₄), and the solvent was evaporated under reduced pressure. The residue was chromatographed (SiO₂, heptane/ethyl acetate, 95:5) to give the dibromide C (5.36 g, 86%) as an orange liquid; R_{f} (heptane-ethyl acetate, 8:2), 0.82. δ_{H} (400 MHz; CDCl₃): 3.6 (2H, m), 3.45 (2H, m), 2.85 (2H, m), 2.1 (2H, m), 1.7 (2H, m; δ_{c}(⁴⁰⁰ MHz; CDCl₃): 39.70, 33.79, 23.95.

Synthesis of Compound 1

To a cooled (0° C.) suspension of potassium hydride (1.58 g, 39.5 mmol) (previously washed 3 times with pentane) in tetrahydrofuran (22 mL) was added, under an argon atmosphere, a solution of methyl methylthiomethyl sulfoxide (1.36 mL, 13.04 mmol, previously dried over molecular sieves for 3 hours) in tetrahydrofuran (3 mL) over 1 hour. After stirring for a further 30 minutes, a solution of C (3.17 g, 13.1 mmol) in THF (2 mL) was added, at 0° C., over 1 hour. The reaction mixture was then allowed to warm up to room temperature and was stirred overnight. The mixture was quenched by addition of aqueous ammonium chloride (6 mL, 25%). After 10 minutes, the solid was filtered off and the filtrate concentrated. The residue was taken up in ether (20 mL) and 9N sulfuric acid (0.05 mL) was added. After stirring for 30 hours, saturated sodium hydrogen carbonate was added. The ether phase was separated and concentrated to 5 mL. Saturated sodium hydrogen sulphite (1.5 g) solution was added and the mixture stirred for 30 minutes. The phases were separated. The ethereal phase was stirred for further 30 minutes with a saturated sodium hydrogen sulphite (0.5 g) solution. The phases were separated and the collected aqueous phases were treated with aqueous sodium hydroxide (5 mL, 20%) and extracted with ether. The ether phase was dried (MgSO₄) and evaporated under reduced pressure to give 4 as a yellow liquid (0.16 g, 11%). δ_{H} (400 MHz; CDCl₃): 3.0 (2H, m), 2.15-2.45 (6H, m), 1.65 (2H, m).

### EXAMPLE 1

Synthesis of:

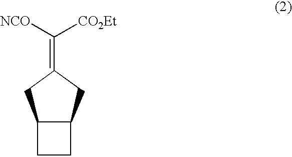

Ketone (1) (199.3 mmol), ethyl cyanoacetate (21.2 ml, 199.3 mmol), ammonium acetate (15.4 g, 199.3 mmol) and glacial acetic acid (11.4 ml, 199.3 mmol) were refluxed in toluene (250 ml) using a Dean-Stark trap. After 8 h, the mixture was allowed to cool and diluted with ethyl acetate (400 ml), washed with water (3×150 ml), brine and dried (MgSO₄). The solvent was evaporated under reduced pressure. The residue was chromatographed (SiO₂, heptanelethyl acetate, 95:5 to 7:3) to give cyano-ester (2) (31.95 g, 78%); R_{f} (heptane-ethyl acetate, 8:2) 0.40; νₘₐₓ(film)/cm⁻¹ 2226 (CN), 1727 (C═O), 1614 (C═C); δ_{H}(400 MHz; CDCl₃) 4.29 (2H, q, J 7.1, CO₂CH₂Me), 3.34 (1H, d, J 20), 3.08-2.88 (5H, m), 2.30-2.18 (2H, m), 1.60-1.51 (2H, m), 1.36 (3H, t, J7.1, CO₂CH₂Me); m/z (Cl⁻) 204 (M−H, 100%).

### EXAMPLE2

Synthesis of:

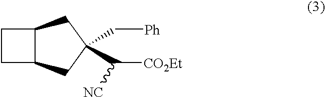

Cyanoester (2) (12.0 g, 59 mmol) in THF (50 ml) was added over 2 h to a stirring solution of benzylmagnesium chloride (117 ml of a 1M solution in ether, 117 mmol) in THF (300 ml) at 15° C. under argon. After allowing the solution to warm to room temperature the mixture was stirred for a further 1 h and then the mixture was cooled to 15° C. and quenched by addition of saturated ammonium chloride solution (100 ml). The mixture was allowed to warm to room temperature, and dilute hydrochloric acid (300 ml) was added. The aqueous layer was extracted with ethyl acetate (2×300 ml). The combined organic layers were washed with brine, dried (MgSO₄) and the solvent was evaporated under reduced pressure to give a 1:1 mixture of diastereomeric cyano-esters (3) (19.85 g, >100% crude yield); R_{f}(heptane-ethyl acetate, 9:1) 0.25; νₘₐₓ(film)/cm⁻¹ 2246 (CN), 1741 (C═O); m/z (Cl⁻) 296 (M−H, 100%); (Cl⁺) 298 (M+H, 90%).

### EXAMPLE 3

Synthesis of:

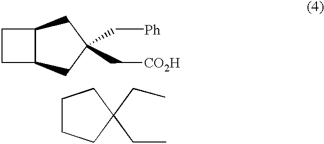

The mixture of diastereomeric cyano-esters (3) (17.39 g, 59 mmol) and potassium hydroxide (19.67 g, 351 mmol) were heated to 160° C. in ethylene glycol (400 ml) for 38 h. After this time, the mixture was allowed to cool and dilute hydrochloric acid (300 ml) was added carefully. The mixture was extracted with ethyl acetate (3×200 ml) and the combined organic fractions were washed with brine, dried (MgSO₄) and the solvent was evaporated under reduced pressure. The residue was chromatographed (SiO₂, heptanelethyl acetate, 8:2) to give the single diastereomeric acid (4) (15.96 g, 95%); R_{f}(heptane-ethyl acetate, 1:1) 0.67; νₘₐₓ(film)/cm⁻¹ 1703 (C═O); δ_{H}(400 MHz; CDCl₃) 7.30-7.17 (5H, m, Ph), 2.84 (2H,m), 2.55 (2H, s, CH₂Ph), 2.44 (2H, s, CH₂CO₂H), 2.29 (2H, m), 2.02 (2H, dd, J 13.2, 8.3), 1.66 (2H, m), 1.50 (2H, dd, J 12.7, 5.2); m/z (Cl⁻) 243 (M−H, 55%);

### EXAMPLE 4

Synthesis of:

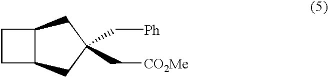

Trimethylsilyldiazomethane (43.2 ml of a 2M solution in hexane, 86.4 mmol) was added dropwise to a stirring solution of acid (4) (17.55 g, 72 mmol) in a mixture of toluene (120 ml) and methanol (50 ml) at 10° C. under argon over 30 minutes. The mixture was allowed to warm to room temperature and stirred for 1 h. The solvent was removed under reduced pressure and the residue was taken up in ethyl acetate (300 ml), washed with saturated sodium hydrogen carbonate (300 ml), dilute hydrochloric acid (300 ml), brine and dried (MgSO₄). The solvent was evaporated under reduced pressure to give ester (5) (17.08 g, 92%); R_{f}(heptane-ethyl acetate, 9:1) 0.51; νₘₐₓ(film)/cm⁻¹ 1737 (C═O); δ_{H}(400 MHz; CDCl₃) 7.39-7.15 (5H, m, Ph), 3.71 (3H, s, OMe), 2.81 (2H, m), 2.51 (2H, s, CH₂CO₂Me), 2.39 (2H, s, CH₂Ph), 2.26 (2H, m), 1.97 (2H, dd, J 13.3, 8.4), 1.64 (2H, m), 1.47(2H, dd, J 12.5,5.1)

### EXAMPLE 5

Synthesis of:

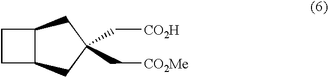

Ester (5) (10.08 g, 39 mmol) and sodium periodate (117 g, 55 mmol) were stirred together in ethyl acetate (58 ml), acetonitrile (58 ml) and water (512 ml) for 5 minutes. The mixture was cooled to 0° C. and ruthenium (III) chloride (0.162 g, 0.8 mmol) was added to the reaction mixture. The reaction was allowed to warm to room temperature and stirred for 24 h with intermittent cooling with an ice bath to control the exotherm. Diethyl ether (100 ml) was added and the mixture was stirred for 40 minutes. The mixture was poured onto dilute hydrochloric acid and extracted with ethyl acetate (2×300 ml). The combined organic fractions were washed with brine, dried (MgSO₄) and the solvent was evaporated under reduced pressure. The residue was purified by chromatography (SiO₂, heptane to 8:2 heptane/ethyl acetate) to give the acid (6) (6.21 g, 66.2%); R_{f}(heptane-ethyl acetate, 1:1) 0.47; νₘₐₓ(film)/cm⁻¹ 1737 (C═O), 1704 (C═O); δ_{H} (400 MHz; CDCl₃) 3.71 (3H, s, OMe), 2.80-2.71 (4H, m), 2.33 (2H, s), 2.26 (2H, m), 2.07 (2H, m), 2.05 (2H, s), 1.64 (2H, m), 1.54 (2H, dd, J 13.2, 5.2); m/z (Cl⁻¹) 225 (M−H), (CIP⁺) 227 (M+H).

### EXAMPLE 6

Synthesis of:

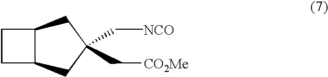

Diphenylphosphoryl azide (3.66 g, 17 mmol), triethylamine (2.43 g, 17.5 mmol), and acid (6) (3.8 g, 16.8 mmol) were refluxed in toluene (50 ml) for 1.25 h. The mixture was allowed to cool and diluted with ethyl acetate (200 ml). The resulting solution was washed with saturated aqueous sodium hydrogen carbonate (200 ml), brine, and dried (MgSO₄). The solvent was removed under reduced pressure to give the isocyanate (7) which was used without further purification (3.75 g, 100%); R_{f}(heptane-ethyl acetate, 9:1) 0.39; νₘₐₓ (film)/cm⁻¹ 2266 (NCO), 1733 (C═O); δ_{H} (400 MHz; CDCl₃) 3.69 (3H, s, OMe), 3.17 (2H, s, CH₂NCO), 2.69 (2H, m), 2.58 (2H, s, CH₂CO₂Me), 2.24 (2H, m), 1.94 (2H, m), 1.65 (2H, m), 1.65 (2H, m), 1.56 (2H, dd, J 12.9, 4.6).

### EXAMPLE 7

Synthesis of:

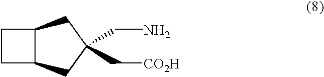

The isocyanate (7) (9.88 g, 45 mmol) and 6N hydrochloric acid (100 ml) were refluxed at 130° C. for 18 h. The mixture was allowed to cool, diluted with water (200 ml) and extracted with dichloromethane (2×200 ml). The aqueous was concentrated to an orange solid and washed with acetonitrile to give the hydrochloride salt of compound (8) (7.10 g, 73%); δ_{H}(400 MHz; d₆-DMSO) 7.88 (2H, br s, NH₂), 2.67 (4H, s), 2.60 (2H, s), 2.22-2.11 (2H, m), 1.94 (2H, dd, J 13.5, 8.0), 1.62 (2H, m), 1.52 (2H, dd, J 13.5, 4.9); m/z (ES⁺) 184 (M+H, 100%); LCMS (Prodigy ODS3 (3μ) 150 mm ×4.6 mmid column, 20-100% Acetonitrile+0.1% formic acid) Retention Time=4.44 min, 100% purity.

The following Examples 8, 9 and 10 illustrate the use of an allyl Grignard reagent in step (ii) and thus involve an allyl compound in steps (iii) and (iv) (“allyl route”).

### EXAMPLE 8

Synthesis of:

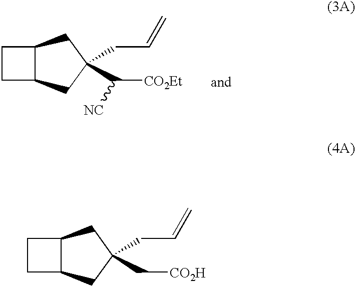

Compound (3A) can be made in two different ways and this affects the yield of compound (4A) as purification is not carried out after synthesis of (3A):

Method A (copper (I) cyanide route)

Allylmagnesium bromide (32.2 ml of a 1 M solution in diethyl ether, 32.2 mmol) was added dropwise to a stirring suspension of copper (I) cyanide (1.44 g pre-dried under vacuum, 16.1 mmol) in THF (60 ml) at 0° C. under argon. After 45 mins, the mixture was cooled to −78° C. and cyanoester (2) (produced as in Example 1) (3.0 g, 14.62 mmol) in THF (30 ml) was added over 1 h. After stirring for a further 1 h, the mixture was quenched by addition of saturated basic ammonium chloride (50 ml of a solution of saturated ammonium chloride with 10% [v/v] concentrated ammonia added). After warming to room temperature, diethyl ether (200 ml) was added and the organic layer was further washed with saturated basic ammonium chloride (3×100 ml) until the aqueous layer was no longer blue. The organic layer was washed with brine, dried (MgSO₄) and the solvent was removed under reduced pressure to give a mixture of diastereomeric mixture of cyanoesters (3A); R_{f}(heptane-ethyl acetate, 7:3) 0.54; νₘₐₓ(film)/cm⁻¹ 2247 (CN), 1742 (C═O); m/z (Cl³¹ ) 246 (M−H, 100%).

The mixture of diastereomeric cyano-esters (3A) (approx 14.6 mmol) and potassium hydroxide (4.92 g, 87.7 mmol) were heated to 160° C. in ethylene glycol (400 ml) for 4 days. After this time, the mixture was allowed to cool and dilute hydrochloric acid (300 ml) was added carefully. The mixture was extracted with ethyl acetate (3×200 ml) and the combined organic fractions were washed with brine, dried (MgSO₄) and the solvent was evaporated under reduced pressure. The residue was chromatographed (SiO₂, heptane/ethyl acetate, 8:2) to give the single diastereomeric acid (4A) (1.97 g, 69%); R_{f}(heptane-ethyl acetate, 1:1) 0.67; νₘₐₓ(film)/cm⁻¹ 1705 (C═O); δ_{H}(400 MHz; CDCl₃) 5.78 (1H, ddt, J 17.1, 10.0, 7.6, CH₂CH═CH_{A}H_{B}), 5.09-4.98 (2H, m, CH₂CH═CH_{A}H_{B}), 2.69 (2H, m), 2.50 (2H, s, CH₂CO₂H), 2.17 (2H, m), 2.01-1.93 (4H, m), 1.64 (2H, m), 1.53 (2H, dd, J 12.8, 5.1); m/z (Cl⁺) 195 (M+H) 100%);

Method B (Dimethylzinc Method)

Allylmagnesium bromide (13.4 ml of a 1 M solution in diethyl ether, 13.4 mmol) was added to a stirring solution of dimethylzinc (6.7 ml of a 2M solution in toluene, 13.4 mmol) in THF (50 ml) at 0° C. under argon. After 20 mins, the mixture was cooled to −78° C. and cyanoester (2) (produced as in Example 1) (2.5 g, 12.18 mmol) in THF (30 ml) was added dropwise over 1 h. After stirring for a further 1 h, the mixture was quenched by careful addition of saturated ammonium chloride solution (30 ml). After warming to room temperature, dilute hydrochloric acid (100 ml to solubilise the zinc salts) was added and the mixture was extracted with diethyl ether (3×150 ml). The combined organic fractions were washed with brine, dried (MgSO₄) and the solvent removed under reduced pressure to give the diastereomeric mixture of cyanoesters (3A).

The mixture of diastereomeric cyano-esters (3A) (approx 12.2 mmol) and potassium hydroxide (4.1 g, 73.1 mmol) were heated to 160° C. in ethylene glycol (400 ml) for 4 days. After this time, the mixture was allowed to cool and dilute hydrochloric acid (300 ml) was added carefully. The mixture was extracted with ethyl acetate (3×200 ml) and the combined organic fractions were washed with brine, dried (MgSO₄) and the solvent was evaporated under reduced pressure. The residue was chromatographed (SiO₂, heptane/ethyl acetate, 8:2) to give the single diastereomeric acid (4A) (2.1 g, 89%).

### EXAMPLE 9

Synthesis of:

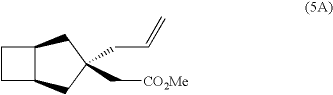

Trimethylsilyldiazomethane (13 ml of a 2M solution in hexane, 25 mmol) was added dropwise to a stirring solution of acid (4A) (4.07 g, 21 mmol) in a mixture of toluene (40 ml) and methanol (10 ml) at 5 to 15° C. under argon over 30 minutes. The mixture was allowed to warm to room temperature and stirred for 1 h. The solvent was removed under reduced pressure and the residue was taken up in ethyl acetate (300 ml), washed with saturated sodium hydrogen carbonate (300 ml), dilute hydrochloric acid (300 ml), brine and dried (MgSO₄). The solvent was evaporated under reduced pressure to give ester (5A) (4.22 g, 96.5%); R_{f}(heptane-ethyl acetate, 9:1) 0.44; νₘₐₓ(film)/cm⁻¹ 1738 (C═O); δ_{H}(400 MHz; CDCl₃) 5.78 (1H, ddt, J 17.1, 10.0, 7.3, CH₂CH═CH_{A}H_{B}), 5.07-4.97 (2H, m, CH₂CH═CH_{B}), 3.67 (3H, s, OMe), 2.69 (2H, m), 2.46 (2H, s, CH₂CO₂H), 2.24 (2H, m), 1.95-1.90 (4H, m), 1.65 (2H, m), 1.50 (2H, dd, J 12.5, 5.1); m/z (Cl⁺) 209 (M+H, 100%);

### EXAMPLE 10

Synthesis of:

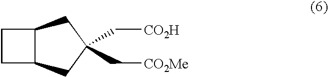

Ester (5A) (4.22 g, 20 mmol) and sodium periodate (18.23 g, 85 mmol) were stirred together in ethyl acetate (31 ml), acetonitrile (31 ml) and water (270 ml) for 5 minutes. The mixture was cooled to 5° C. and ruthenium (III) chloride (0.044 g, 0.4 mmol) was added to the reaction mixture. The reaction was allowed to warm to room temperature and stirred for 24 h with intermittent cooling with an ice bath to control the exotherm. Diethyl ether (100 ml) was added and the mixture was stirred for 40 minutes. The mixture was poured onto dilute hydrochloric acid and extracted with ethyl acetate (2×400 ml). The combined organic fractions were washed with brine, dried (MgSO₄) and the solvent was evaporated under reduced pressure. The residue was purified by chromatography (SiO₂, heptane to 8:2 heptane/ethyl acetate) to give the acid (6) (3.8 g, 83%).

The acid (6) can then be converted to the isocyanate (7) and the desired produce (8) as in Examples 6 and 7.

1. A process for preparing (1α,3α,5α)(3-aminomethyl-bicyclo[3.2.0]hept-3-yl)-acetic acid, or an acid addition salt thereof, which comprises the following steps:
(i) condensing a cyclic ketone (1) with an alkyl cyanoacetate to form a cyanoester (2): 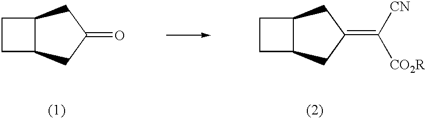
in which R is an alkyl group having 1 to 6 carbon atoms;
(ii) reacting the cyanoester (2) with an arylalkyl or alkenyl grignard reagent to form a cyanoester (3): 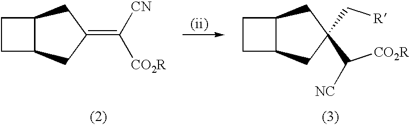
in which R′ is a phenyl or phenyl-C₁-C₄ alkyl group or a C₂-C₆ alkenyl group;
(iii) removing the cyano group of the cyanoester (3) by reaction with a base to form a carboxylic acid (4): 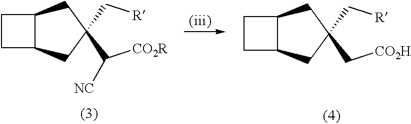
(iv) converting the carboxylic acid (4) to its alkyl ester (5): 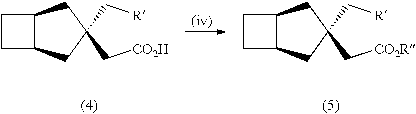
in which R″ is an alkyl group having 1 to 6 carbon atoms;
(v) oxidising the alkyl ester (5) to form the acid (6): 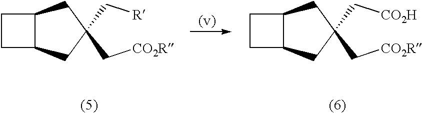
(vi) converting the carboxylic acid group of the acid (6) to an isocyanate group, thereby forming the compound (7): 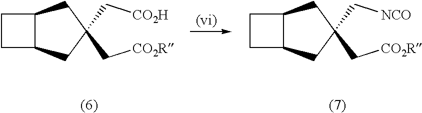
and
(vii) hydrolysing the isocyanate and ester groups of compound (7) to form the desired compound (1α,3α,5α)(3-aminomethyl-bicyclo[3.2.0]hept-3-yl)-acetic acid (8), or an acid addition salt thereof:

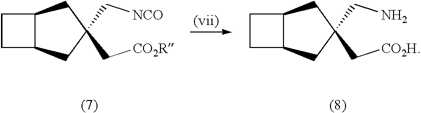

2. A process according to claim 1, in which the arylalkyl Grignard reagent used in step (ii) is a benzyl Grignard reagent.

3. A process according to claim 2, in which the benzyl Grignard reagent is benzylmagnesium chloride, benzylmagnesium bromide or benzylmagnesium iodide.

4. A process according to claim 1, in which the alkenyl Grignard reagent used in step (ii) is a vinyl, allyl or 2-butenyl Grignard reagent.

5. A process according to claim 4, in which the Grignard reagent is vinyl lithium, allylmagnesium chloride, allylmagnesium bromide or 2-butenylmagnesium chloride.

6. A process according to claim 4, in which step (ii) is carried out in the presence of a dialkylzinc or a copper (I) salt.

7. A process according to claim 5, in which step (ii) is carried out in the presence of a dialkylzinc or a copper (I) salt.
